(19) United States
(12) Patent Application Publication
(10) Pub. No.: US 2021/0366122 A1
(43) Pub. Date: Nov. 25, 2021

(54) Method and apparatus for segmenting G-banded adhered chromosome based on geometrical characteristic and regional fusion, and chromosome karyotype analysis device

(71) Applicants: HUNAN ZIXING INTELLIGENT MEDICAL TECHNOLOGY CO., LTD, Changsha, Hunan (CN); HUNAN GUANGXIU FUTURE MEDICAL HEALTH INDUSTRY GROUP CO. LTD., Changsha, Hunan (CN)

(72) Inventors: Shengri FENG, Changsha, Hunan (CN); Ge LIN, Changsha, Hunan (CN); Yi LI, Changsha, Hunan (CN); Guangxiu LU, Changsha, Hunan (CN); Yang MU, Changsha, Hunan (CN); Yueqiu TAN, Changsha, Hunan (CN); Yufeng CAI, Changsha, Hunan (CN); Zixing CAI, Changsha, Hunan (CN)

(21) Appl. No.: 17/043,659

(22) PCT Filed: Oct. 22, 2018
(86) PCT No.: PCT/CN2018/111249

§ 371 Date: Sep. 30, 2020

(30) Foreign Application Priority Data
Jun. 20, 2018 (CN) 201810636329.5

(51) Int. Cl. G06T 7/11; G06T 7/00; G01N 33/48; G06T 7/60

(52) U.S. Cl. CPC G06T 7/11; G06T 7/60; G01N 33/48; G06T 7/0012

(57) ABSTRACT

Provided are a method and an apparatus for segmenting a G-banded adhered chromosome based on a geometrical characteristic and regional fusion, and a chromosome karyotype analysis device. The method first extracts concave points of an outline of an adhered chromosome region, then cuts an adhered chromosome image via a cutting line formed by the concave points in pairs, and at last carries out a fusion operation on a local cut region and selects a most appropriate region as a final single chromosome region. The method implements automatic segmentation of an adhered chromosome and improves the chromosome karyotype analysis efficiency.

### CROSS-REFERENCE TO RELATED APPLICATIONS

This application is the national stage entry of PCT/CN2018/111249, filed on Oct. 22, 2018, which are incorporated by reference in their entirety herein.

### TECHNICAL FIELD

The present invention relates to the field of image processing, and in particular to a method and an apparatus for segmenting a G-banded adhered chromosome based on a geometrical characteristic and regional fusion, and a chromosome karyotype analysis device.

### BACKGROUND

In relevant reproduction and genetics specialized hospitals, chromosome karyotype analysis is an important medical diagnostic means. At present, along with the development of a computer image processing technology, various novel image processing algorithms for a chromosome image emerge endlessly. All these methods are basically intended to improve and promote an original software system that processes the chromosome image with a main reliance on human assistance.

Before analysis of a chromosome karyotype, the segmentation for an image of an adhered chromosome is still a difficult problem. Due to softness of the chromosome, the chromosome after being produced into a flake has an adhesion diversity. Base forms of the adhered chromosome approximately include: a lightly-touched adhesion, a serious adhesion, an intersection, an overlap, and a mixed adhesion consisting of these base forms for multiple chromosomes. Currently, the software system used by most specialized hospitals basically still need the human assistance for segmentation. In a segmentation research for adhesion of the chromosome, different segmentation methods are pushed forward by relevant scholars. Among them, a method for first classifying an adhered form of the chromosome and then segmenting the chromosome is included, and such a method first divides the form of the chromosome into a “T” form, an “X” form, an “H” form and other adhered forms, then extracts characteristics of different forms one by one and segmenting; a method for carrying out eroding and dilating operations on the adhered chromosome based on a mathematical morphological method, obtaining a morphological nucleus of the chromosome via excessive erosion and finally obtaining a single chromosome via reverse dilation is included; and a method for carrying out adhesion segmentation on the chromosome in combination with a geometric morphological characteristic and a Support Vector Machine (SVM) classifier is further included. Concepts for implementing the methods pushed forward by predecessors are different, but a general direction is to depend on extraction of a geometric characteristic of the chromosome. For example, to describe the geometric characteristic of the form of the adhered chromosome, most scholars extract a chromosome outline first, and then search, according to a curvature change of an outline curve, concave points formed by adhesion. A method for calculating the curvature change of the outline curve approximately includes: a Freeman chain code method, a curve fitting method, etc. In brief, the segmentation of the adhered chromosome with the reliance on the geometric characteristic is mainly determined by a robustness for describing characteristics of the chromosome; and moreover, these characteristics are basically further determined by an imaging quality of a specific chromosome and a relevant experience-based judgment. Additionally, research achievements of the above predecessors for a flake production type of the chromosome are also different. For instance, the excessive erosion method has a good effect to a Q-banded chromosome but a poor segmentation effect to the G-banded chromosome involved in the present invention, which is mainly attributed to that a kinetochore of the G-banded chromosome is obvious to become difficult to obtain an appropriate morphological nucleus of the chromosome.

To sum up, for a chromosome G-banded metaphase greyscale image, the present invention provides a method for segmenting a G-banded adhered chromosome based on the geometrical characteristic and regional fusion.

### SUMMARY

In order to improve the intellectualization of a chromosome karyotype analysis system, solving a segmentation problem of an adhered chromosome becomes crucial. In view of this, a method and an apparatus for segmenting a G-banded adhered chromosome based on a geometrical characteristic and regional fusion, and a chromosome karyotype analysis device are provided.

A first aspect of the present invention is implemented via the following solutions.

A method for segmenting a G-banded adhered chromosome based on a geometrical characteristic and regional fusion includes the following steps.

At Step 1: a chromosome G-banded metaphase grayscale image subjected to noise removal processing is read.

At Step 2: all chromosome communicated regional images of the image in the step 1 are extracted and stored to an image set AR.

At Step 3: chromosome communicated regional image sets Si and Ad are created, wherein the image set Si is used for storing a single chromosome image, and the image set Ad is used for storing a non-single chromosome image.

At Step 4: all chromosome communicated regional images in the image set AR in the step 2 are traversed, a regional image meeting a condition I or a condition II or a condition III is viewed as a single chromosome and stored to the single chromosome image set Si, or otherwise, storing to the image set Ad of the non-single chromosome image, wherein the image set Si and the image set Ad are as mentioned in the step 3.

The condition I refers to that the number of concave points of an outline of a chromosome communicated regional image is smaller than a parameter T3.

The condition II refers to that a ratio of an independent area of the chromosome communicated regional image to an area of a convex hull is greater than a parameter T4.

The condition III refers to that a skeleton line of the chromosome communicated regional image has two end points, and with line fitting of a least square method on a skeleton coordinate sequence, a residual standard difference of the skeleton coordinate sequence is counted to be smaller than a parameter T5.

At Step 5: the chromosome regional images in the set Si in the step 4 are traversed, an average width W for chromosome regions meeting the condition II or the condition Ill are calculated, and a maximum value Wₘₐₓ and a minimum value Wₘᵢₙ of the average width for all chromosome regions meeting the condition II or the condition Ill are counted.

At Step 6: the chromosome regional images in the set Ad in the step 4 are traversed; and for any image P in the set Ad, a coordinate sequence of an outline of a chromosome region of the P is extracted, the number N of concave points of the outline of the P is calculated, and a coordinate set of the concave points is represented by the following formula:

PIT={(xᵢ,yᵢ)| the i is an integer between [1,N]}

Where, the N is the number of concave points of the outline of the image P; the PIT is the coordinate set of the concave points, PIT(i) is recorded as a coordinate of an ith concave point and the i is a subscript index value; the xᵢ represents a horizontal coordinate of the ith concave point; and the yᵢ represents a vertical coordinate of the ith concave point.

At Step 7: the concave points of the outline of the image P in the step 6 are combined in pairs to form a cutting line, wherein C_{N}² cutting lines are provided in total, and a set formed by these cutting lines is represented by the following formula:

CUT={(PIT(i),PIT(j))|i≠j and is the integer between [1,N]}

Where, the N is the number of concave points of the outline of the image P in the step 6; the CUT is the set of the cutting lines, and CUT(k) is recorded as a kth cutting line; the PIT(i) and the PIT(j) are coordinates of concave points on two ends of the cutting line; and the i, the j and the k are all the subscript index values.

At Step 8: the cutting lines are screened, a cutting line not meeting the condition IV is removed, and a set of remaining cutting lines is recorded as CUT′, wherein CUT′(i) is recorded as an ith effective cutting line, the i is the subscript index value, and the condition IV is as follows: any two concave points of the outline of the chromosome communicated regional image need to meet that a cutting line segment formed by the two concave points does not pass through a background of the regional image, and a double of a linear distance for the two concave points is smaller than a shortest length for the two concave points along the outline.

At Step 9: since any adhered chromosome regional image P in the set Ad and the effective cutting line set CUT′ thereof are calculated in the step 6 to the step 8, an adhered chromosome segmentation strategy is carried out on the image P.

At Step 10: if the set Ad is not null, a next adhered chromosome regional image is segmented continuously, and the steps 6-9 are repeated; or otherwise, an adhered chromosome segmentation procedure is ended.

As a further improvement, in the step 3, the non-single chromosome is an adhered chromosome.

As a further improvement, a method for calculating the average width for the chromosome regions in the step 5 is as follows.

1) Variables K=5 and STEP=2 are initialized, wherein the K represents a serial number of an index value, and the STEP represents an index step length.

2) Outlines and skeleton coordinate sequences of the chromosome regions are respectively extracted in sequence, wherein the skeleton coordinate sequences are recorded as S and S(i) is recorded as an ith skeleton point coordinate.

3) i=K+1 is initialized.

4) If the number of points in the skeleton coordinate sequences S is smaller than 2*K+1, the step 5) is executed; or otherwise, the steps 6) to 9) are executed.

5) line fitting is carried out on all coordinates in the skeleton coordinate sequences S with a least square method to obtain a fitted line, a slope a of a perpendicular line perpendicular to the fitted line is calculated, a distance d between two points where a straight line passing through a midpoint of the S and having the slope of a is intersected with the outlines of the chromosome regions is calculated, the distance serves as the average width W for the chromosome regions and the process is ended.

6) Segmental line fitting is carried out on (i−K)th to (i+K)th points in the S with the least square method to obtain a fitted line, a slope a of a perpendicular line perpendicular to the fitted line is calculated, a distance d between two points where a straight line passing through a S(i) and having the slope of a is intersected with the outline of a chromosome region is calculated, and the distance serves as a width of a chromosome region at the point S(i).

7) The i is modified as i=i+STEP.

8) The step 6) is repeated, till i+K is greater than the number of points in the S. 9) An average value W for widths of the chromosome regions is calculated at last, and the process is ended.

As a further improvement, a method for calculating the concave points of the chromosome regions in the step 6 is as follows.

1) As mentioned in the step 6, for any non-single chromosome regional image P, a coordinate sequence of an outline of the non-single chromosome region is extracted first and recorded as B, and an ith outline coordinate is recorded as B(i), wherein the i is the subscript index value.

2) Variables K=2, MAXSTEP=7 and i=1 are initialized, wherein the MAXSTEP represents a maximum step length.

3) The coordinate sequence of the outline of the chromosome regions are traversed in sequence; and for a coordinate position of an ith outline, a concave angle θ at the coordinate position of the ith outline is calculated, with a cosine value as follows:

$$cos ( θ ) = \frac{\overline{B ( i ) B ( i - K )} • \overline{B ( i ) B ( i + K )}}{\vert \overline{B ( i ) B ( i - K )} \vert \overline{\vert B ( i ) B ( i + K )} \vert}$$

Where, the B̅(̅i̅)̅B̅(̅i̅−̅K̅)̅ represents a vector from an ith point B(i) to an (i−K)th point B(i−K) on the outline; and the B̅(̅i̅)̅B̅(̅i̅+̅K̅)̅ represents a vector from the ith point B(i) to an (i+K)th point B(i+K) on the outline.

4) An outline point having the concave angle θ<T1 and a midpoint between B(i−K) and B(i+K) out of the chromosome region is marked as a candidate concave point.

5) i=i+1; if the i is greater than the number of outline points, the step 6) is executed; or otherwise, the steps 3) and 4) are continued.

6) K=K+1; if the K is greater than the MAXSTEP, the step 7) is executed; or otherwise, i=1 is re-initialized, and the steps 3), 4) and 5) are continued.

7) The outline point between two candidate concave points with a distance smaller than 5 pixels along the outline is marked as the candidate concave point.

8) A midpoint of a candidate concave point segment is determined: two candidate concave points having the distance of 1 pixel along the outline are considered as being in a same concave point segment, and the midpoint of the concave point segment is found to serve as a final outline concave point.

As a further improvement, in the step 1), the method for extracting the coordinate sequences of the outlines is obtained with a relevant Application Program Interface (API) function in an open source computer vision library OpenCV.

As a further improvement, in the step 9, a method of the adhered chromosome segmentation strategy is as follows.

1) As mentioned in the steps 6-8, for any adhered chromosome regional image P, the effective cutting line set CUT′, and the number n of cutting lines are calculated.

2) Two sets M and C having a length of n elements are respectively created, wherein the type of each element in the M is the image, the type of each element in the C is the cutting line, and the elements in the two sets are initialized as 0; the variable i=1 is initialized, and two image sets Si′ and Ad′ are initialized to be null, wherein the Si′ represents a segmented single chromosome image set, and the Ad′ represents a segmented non-single chromosome image set.

3) The image P is segmented into two portions P_{A} and P_{B} with the cutting line CUT′(i).

4) Whether the P_{A} and the P_{B} simultaneously meet the condition III and a condition V is determined; if yes, the P_{A} and the P_{B} at this time are two segmented single chromosomes respectively, images of the two single chromosomes are stored to an Si′ set and the step 12) is executed; or otherwise, the step 5) is executed, wherein the condition V refers to that the skeleton line of the chromosome communicated regional image has two end points, the average value of the width for the chromosome region of the image is between [c1·Wₘᵢₙ,c2·Wₘₐₓ], and the c1 and the c2 are a correction factor.

5) Whether the P_{A} meets the condition III or the condition V and the P_{B} also meets the condition III or the condition V is determined; if yes, the cutting line at this time is stored to the set C, and the step 7) is executed; or otherwise, the step 6) is executed.

6) Whether either the P_{A} or the P_{B} meets the condition III and the condition V is determined; and if yes, an image meeting the conditions is assigned to a set M.

7) i=i+1 and whether the i is greater than n is determined; if yes, the step 8) is executed; or otherwise, the step is returned continuously to execute the steps 3), 4), 5) and 6).

8) If the set C is not null, a shortest cutting line in the C is a final cutting line, the image P is segmented into two single chromosome portions with the shortest cutting line, the two single chromosome portions are stored to the Si′, and the step 12) is executed; and if the C is null, the step 9) is executed.

9) Whether the number of elements in the set M is equal to 1 is determined; if yes, the unique one element in the set M is the segmented single chromosome portion, an image of the single chromosome is stored to the Si′ set, and the step 12) is executed; or otherwise, the step 10) is executed.

10) Whether the number of elements in the set M is greater than 1 is determined; if yes, a fusion operation in the step 11) is executed; or otherwise, it is indicated that the adhered chromosome regional image P cannot be segmented effectively, and the adhesion segmentation procedure is ended.

11) The fusion operation is carried out on the elements in the set M in pairs. a. Kth and jth image elements M(k) and M(j) (k≠j) in the M are accessed, wherein sizes of the two images are the same as the original adhered image P, and a chromosome region in each image is one portion of the P.

b. An intersection area s1 between the chromosome regions in the M(k) and M(j) images is calculated, wherein the size of the intersection area is represented by the number of intersected pixels in the chromosome regions, and a sufficient and necessary condition for an existence of an intersection between the chromosome regions of the two images is that a pixel corresponding to a same coordinate position in the two images is within the chromosome regions.

c. A minimum value s2 of the area of the chromosome region in each of the M(k) and M(j) images is calculated, wherein the area of each chromosome region refers to the number of pixels in the chromosome region.

d. If

$$\frac{s 1}{s 2} > T 2 ,$$

an image having a small area of the chromosome region between the M(k) and the M(j) is deleted from the set M, or otherwise, any other two image elements in the set M is accessed continuously, and the above steps b, c and d are repeated.

e. After the steps b, c and d are executed completely, remaining elements in the set M are fused single chromosome images, these single chromosome images are stored to the Si′, and the step 12) is executed.

12) The segmented single chromosomes are stored in the set Si′, these chromosome regions are removed from the original chromosome regional image P and then remaining chromosome regional images are stored to the Ad′.

13) The chromosome regions in the Ad′ are viewed as the non-single chromosomes, and the steps 6-9 are repeated continuously.

As a further improvement, the T1=2.5, T2=0.78, T3=2, T4=0.8, T5=2.0, and correction factors c1=0.9, c2=1.1.

According to a second aspect of the present invention, an apparatus for segmenting a G-banded adhered chromosome based on a geometrical characteristic and regional fusion is provided. The apparatus is configured to store or run a module, or the module is a constituent part of the apparatus; the module is a software module; one or more pieces of the software are provided; and the software module is configured to execute the above-mentioned method for segmenting the G-banded adhered chromosome based on the geometrical characteristic and the regional fusion.

According to a third aspect of the present invention, a chromosome karyotype analysis device is provided, which includes an apparatus for segmenting an adhered image and an apparatus for carrying out karyotype analysis on a segmented chromosome; and the apparatus for segmenting the adhered image is the above-mentioned apparatus for segmenting the G-banded adhered chromosome based on the geometrical characteristic and the regional fusion.

To sum up, the method provided by the present invention is novel and simple. A general concept of the method is to first extract concave points of an outline of an adhered chromosome region, then cut an adhered chromosome image via a cutting line formed by the concave points in pairs, and at last carry out a fusion operation on a local cut region and select a most appropriate region as a final single chromosome region. A final segmentation effect of the method is determined by a characteristic explanation on the chromosome region in the present invention, that is, a condition I to a condition V. It is proved by an experiment that the more complete the characteristic explanation on the chromosome image, the better the effect of the segmentation method of the present invention. Therefore, a subsequent characteristic research on the chromosome region may still serve as a supplementation to the conditions.

The parameters T1, T2, T3, T4, T5 and the correction factors c1, c2 in the above steps are all optimal values obtained by a test, and may also be adjusted and altered according to an actual condition.

### BRIEF DESCRIPTION OF THE DRAWINGS

The accompanying drawings formed into a part of the present invention are described here to provide a further understanding of the present invention. The schematic embodiments and description of the present invention are adopted to explain the present invention, and do not form improper limits to the present invention. In the drawings:

FIG. 1 and FIG. 2 are respectively an original adhered chromosome image and an adhered form with two chromosomes adhered lightly.

FIG. 3 and FIG. 4 are respectively a binary image corresponding to FIG. 1 and FIG. 2.

FIG. 5 and FIG. 6 are respectively an outline image and a concave point marking result corresponding to FIG. 1 and FIG. 2.

FIG. 7 and FIG. 8 are respectively an optimal segmentation results corresponding to FIG. 1 and FIG. 2.

FIG. 9 and FIG. 10 are respectively an original adhered chromosome image, with an adhered form being that three chromosomes are adhered.

FIG. 11 and FIG. 12 are respectively a binary image corresponding to FIG. 9 and FIG. 10.

FIG. 13 and FIG. 14 are respectively an outline image and a concave point marking result corresponding to FIG. 9 and FIG. 10.

FIG. 15 and FIG. 16 are respectively an optimal segmentation result corresponding to FIG. 9 and FIG. 10.

### DETAILED DESCRIPTION OF THE EMBODIMENTS

It is to be noted that the embodiments of the present invention and the characteristics of the embodiments may be combined with each other if there is no conflict. The present invention is described below in detail in combination with the embodiments and the accompanying drawings.

In a first typical implementation manner, a method for segmenting a G-banded adhered chromosome based on a geometrical characteristic and regional fusion is provided. A specific example capable of implementing effective segmentation of the adhered chromosome with the method is as follows.

### Embodiment 1

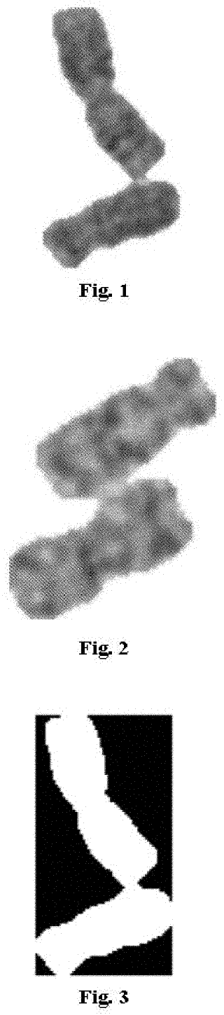

(1) An adhered chromosome image is read as shown in FIG. 1, and recorded as I1.

(2) Binary segmentation is carried out on the I1 to obtain FIG. 3 that is recorded as I2.

(3) Outline extraction and concave point calculation are carried out on the I2 to obtain an outline and a concave point of the I1 chromosome region, as shown in FIG. 5, wherein a line is an outline mark of the chromosome region, and the concave point is marked by a circle.

(4) The I1 is segmented via the segmentation method of the present invention, with a segmentation result as shown in FIG. 7.

### Embodiment 2

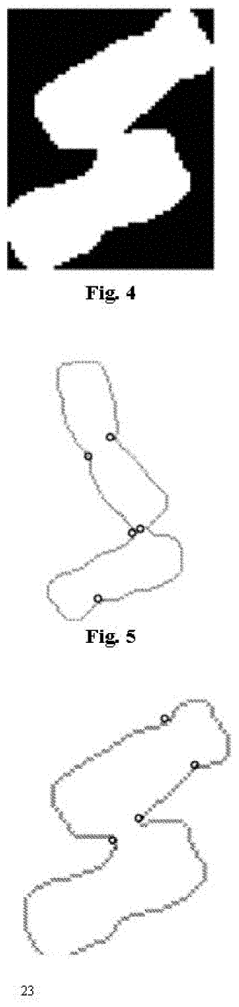

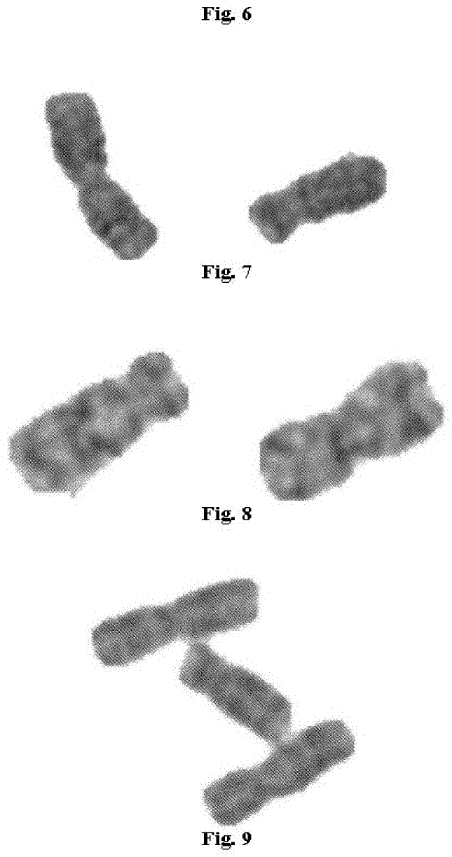

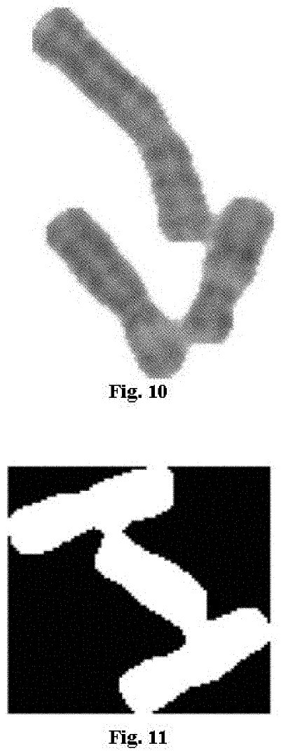

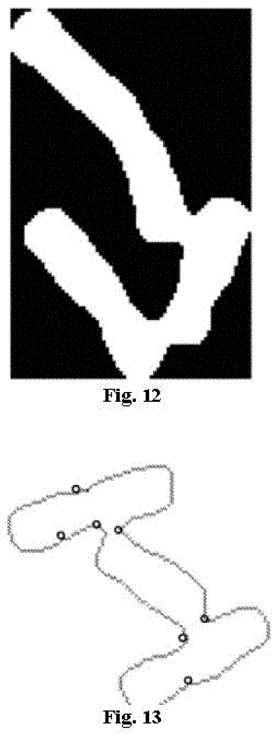

(1) An adhered chromosome image is read, as shown in FIG. 2 in which an adhered form is that two chromosomes are adhered lightly, and recorded as I3.

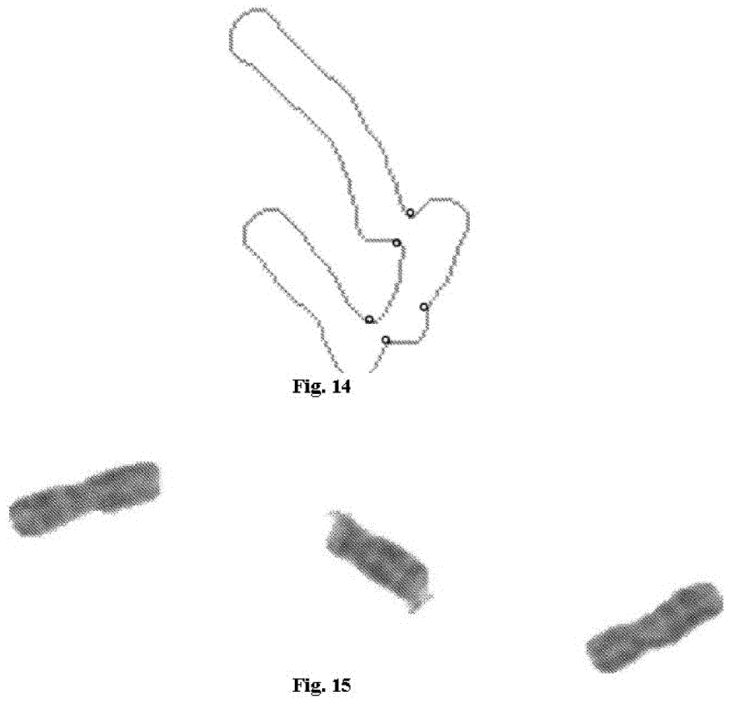

(2) Binary segmentation is carried out on the I3 to obtain FIG. 4 that is recorded as I4.

(3) Outline extraction and concave point calculation are carried out on the I4 to obtain an outline and a concave point of the I2 chromosome region, as shown in FIG. 6, wherein a line is an outline mark of the chromosome region, and the concave point is marked by a circle.

(4) The I2 is segmented via the segmentation method of the present invention, with a segmentation result as shown in FIG. 8.

### Embodiment 3

(1) An adhered chromosome image is read, as shown in FIG. 9 and FIG. 10 in which three chromosomes are adhered, and respectively recorded as I5 and I7.

(2) Binary segmentation is respectively carried out on the I5 and the I7 to obtain FIG. 11 that is recorded as I6, and FIG. 12 that is recorded as I8.

(3) Outline extraction and concave point calculation are carried out on the I6 and the I8 to obtain an outline and a concave point of the I5 and I7 chromosome regions, as shown in FIG. 13 and FIG. 14, wherein a line is an outline mark of the chromosome region, and the concave point is marked by a circle.

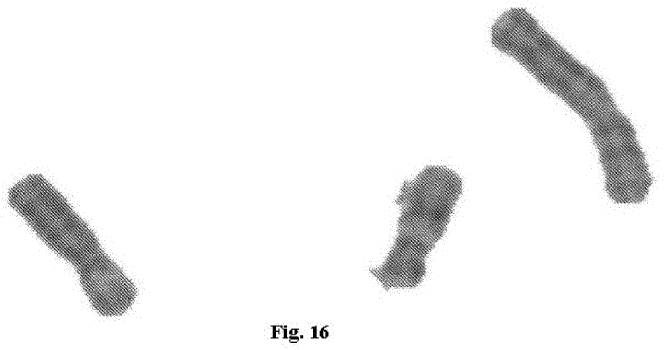

(4) The I5 and the I7 are segmented via the segmentation method of the present invention, with a segmentation result as shown in FIG. 15 and FIG. 16.

The serial numbers of the embodiments of the present invention are merely for description and do not represent a preference of the embodiments.

In the above embodiments of the present invention, the description on each embodiment has its preference, and the part not detailed in some embodiments may be referred to related description on other embodiments.

It is to be noted that, for ease of description, the foregoing method embodiments are described as a series of action combinations. However, a person skilled in the art should understand that the present invention is not limited to the described sequence of the actions, because some steps may be performed in another sequence or performed at the same time according to the present invention. In addition, the person skilled in the art should also appreciate that all the embodiments described in the specification are preferred embodiments, and the related actions and modules are not necessarily mandatory to the present invention.

By means of the above-mentioned descriptions on the implementation manner, the person skilled in the art may clearly understand that the present invention may be implemented by software plus a necessary universal hardware platform, and may also be implemented by hardware, but under most conditions, the former is a better implementation manner. Based on this understanding, the technical solutions in the present invention essentially or the part contributing to the prior art may be embodied in the form of a software product, the computer software product may be stored in a storage medium (such as a Read-Only Memory (ROM)/Read Access Memory (RAM)), and include several instructions for instructing a computing device to execute the methods in the embodiments of the present invention, or instructing a processor to execute the methods in the embodiments of the present invention.

Therefore, according to a second typical implementation manner of the present invention, an apparatus for segmenting a G-banded adhered chromosome based on a geometrical characteristic and regional fusion is provided. The apparatus is configured to store or run a module, or the module is a constituent part of the apparatus; the module is a software module; one or more pieces of the software are provided; and the software module is configured to execute the above-mentioned method for segmenting the G-banded adhered chromosome based on the geometrical characteristic and the regional fusion.

According to a third typical implementation manner of the present invention, a chromosome karyotype analysis device is provided, which includes an apparatus for segmenting an adhered image and an apparatus for carrying out karyotype analysis on a segmented chromosome; and the apparatus for segmenting the adhered image is the above-mentioned apparatus for segmenting the G-banded adhered chromosome based on the geometrical characteristic and the regional fusion.

In the several embodiment provided by the present invention, it should be understood that the disclosed technical content may be implemented via other manners. The described apparatus embodiment is merely exemplary. For example, the unit division is merely logical function division and may be other division in actual implementation. For example, a plurality of units or components may be combined or integrated into another system, or some features may be ignored or not performed. In addition, the displayed or discussed mutual couplings or direct couplings or communication connections may be implemented through some interfaces. The indirect couplings or communication connections between the units or modules may be implemented in electronic or other forms.

The units described as separate parts may or may not be physically separate, and parts displayed as units may or may not be physical units, may be located in one position, or may be distributed on a plurality of network units. Some or all of the units may be selected according to actual needs to achieve the objectives of the solutions of the embodiments.

In addition, functional units in the embodiments of the present invention may be integrated into one processing unit, or each of the units may exist alone physically, or two or more units are integrated into one unit. The integrated units may be implemented in a form of hardware, and may also be implemented in a form of a software functional unit.

When the integrated units are implemented in the form of the software functional unit and sold or used as an independent product, the integrated units may be stored in a computer-readable storage medium. Based on this understanding, the technical solutions in the present invention essentially or the part contributing to the prior art or all or a part of the technical solutions may be embodied in a form of a software product; and the computer software product is stored in a storage medium and includes several instructions for instructing a computing device (which may be a personal computer, a server or a network device or the like) to execute all or a part of steps of the method in each embodiment of the present invention. The foregoing storage medium includes: any medium that can store a program code, such as a U disk, an ROM, an RAM, a mobile hard disk, a magnetic disk, or an optical disc.

From the above descriptions, it may be seen that the above embodiments of the present invention implement the following technical effects: the present invention first extracts concave points of an outline of an adhered chromosome region, then cut an adhered chromosome image via a cutting line formed by the concave points in pairs, and at last carry out a fusion operation on a local cut region and select a most appropriate region as a final single chromosome region. The present invention implements automatic segmentation on the adhered chromosome, and improves the chromosome karyotype analysis efficiency.

The above descriptions are only preferred embodiments of the present invention and are not intended to limit the present invention. For the person skilled in the art, the present invention may have various modifications and changes. Any modification, equivalent replacement, improvement and the like made within a spirit and a principle of the present invention should be included in a protection scope of the present invention.

What is claimed is:

1. A method for segmenting a G-banded adhered chromosome based on a geometrical characteristic and regional fusion comprises the following steps:
step 1: reading a chromosome G-banded metaphase grayscale image subjected to noise removal processing;
step 2: extracting all chromosome communicated regional images of the image in the step 1 and storing to an image set AR;
step 3: creating an image set Si and an image set Ad for the chromosome communicated regional images, wherein the image set Si is used for storing a single chromosome image, and the image set Ad is used for storing a non-single chromosome image;
step 4: traversing all the chromosome communicated regional images in the image set AR in the step 2, viewing a regional image meeting a condition I or a condition II or a condition III as a single chromosome and storing to the image set Si of the single chromosome image, or otherwise, storing to the image set Ad of the non-single chromosome image, wherein the image set Si and the image set Ad are as mentioned in the step 3;
the condition I refers to that the number of concave points of an outline of a chromosome communicated regional image is smaller than a parameter T3;
the condition II refers to that a ratio of an independent area of the chromosome communicated regional image to an area of a convex hull is greater than a parameter T4; and
the condition III refers to that a skeleton line of the chromosome communicated regional image has two end points, and with line fitting of a least square method on a skeleton coordinate sequence, a residual standard difference of the skeleton coordinate sequence is counted to be smaller than a parameter T5;
step 5: traversing the chromosome regional images in the image set Si in the step 4, calculating an average width W for chromosome regions meeting the condition II or the condition III, and counting a maximum value Wₘₐₓ and a minimum value Wₘᵢₙ of the average width for all chromosome regions meeting the condition II or the condition III;
step 6: traversing the chromosome regional images in the image set Ad in the step 4, and for any image P in the image set Ad, extracting a coordinate sequence of an outline of a chromosome region of the P, calculating the number N of concave points of the outline of the P, and representing a coordinate set of the concave points by the following formula:
PIT={(xᵢ,yᵢ)| the i is an integer between [1, N]}
where, the N is the number of concave points of the outline of the image P, the PIT is the coordinate set of the concave points, PIT(i) is recorded as a coordinate of an ith concave point and the i is a subscript index value; the xᵢ, represents a horizontal coordinate of the ith concave point; and the yᵢ, represents a vertical coordinate of the ith concave point;
step 7: combining the concave points of the outline of the image P in the step 6 in pairs to form a cutting line, wherein C_{N}² cutting lines are provided in total, and a set formed by the cutting lines is represented by the following formula:
CUT={(PIT(i),PIT(j))|i≠j and is the integer between [1,N]}
where, the N is the number of concave points of the outline of the image P in the step 6, the CUT is the set of the cutting lines, and CUT(k) is recorded as a kth cutting line, the PIT(i) and the PIT(j) are coordinates of concave points on two ends of the cutting line CUT(k), and the i, the j and the k are all the subscript index values;
step 8: screening the cutting lines, removing a cutting line not meeting the condition IV, and recording a set of remaining cutting lines as CUT′, wherein CUT′ (i) is recorded as an ith effective cutting, line, the i is the subscript index value, and the condition IV is as follows: any two concave points of the outline of the chromosome communicated regional image need to meet that a cutting line formed by the two concave points does not pass through a background of the regional image, and a double of a linear distance for the two concave points is smaller than a shortest length for the two concave points along the outline;
step 9: since any adhered chromosome regional image P in the image set Ad and the effective cutting line set CUT′ thereof are calculated in the step 6 to the step 8, carrying out an adhered chromosome segmentation strategy on the image P; and
step 10: if the image set Ad is not null, segmenting a next adhered chromosome regional image continuously, and repeating the steps 6-9; or otherwise, ending an adhered chromosome segmentation procedure.

2. The method for segmenting the G-banded adhered chromosome based on the geometrical characteristic and the regional fusion as claimed in claim 1, wherein in the step 3, the non-single chromosome is an adhered chromosome.

3. The method for segmenting the G-banded adhered chromosome based on the geometrical characteristic and the regional fusion as claimed in claim 1, wherein a method for calculating the average width for the chromosome regions in the step 5 is as follows:
1) initializing variables K=5 and STEP=2, wherein the K represents a serial number of an index value, and the STEP represents an index step length;
2) respectively extracting outlines and skeleton coordinate sequences of the chromosome regions in sequence, wherein the skeleton coordinate, sequences are recorded as S and S(i) is recorded as an ith skeleton point coordinate;
3) initializing i=K+1;
4) if the number of points in the skeleton coordinate sequences S is smaller than 2*K+1, executing the step 5); or otherwise, executing the steps 6) to 9);
5) carrying out line fitting on all coordinates in the skeleton coordinate sequences S with a least square method to obtain a fitted line, calculating a slope a of a perpendicular line perpendicular to the fitted line, calculating a distance d between two points where a straight line passing through a midpoint of the S and having the slope of a is intersected with the outlines of the chromosome regions, taking the distance as the average width W for the chromosome regions and ending the process;
6) carrying out segmental line fitting on (i−K)th to (i+K)th points in the S with a least square method to obtain a fitted line, calculating a slope a of a perpendicular line perpendicular to the fitted line, calculating a distance d between two points where a straight line passing through a S(i) and having the slope of a is intersected with the outline of a chromosome region, and taking the distance as a width of the chromosome region at the point S(i);
7) modifying the i as i=i+STEP;
8) repeating the step 6), till i+K is greater than the number of points in the S; and
9) calculating an average value W for widths of the chromosome regions at last, and ending the process.

4. The method for segmenting the G-banded adhered chromosome based on the geometrical characteristic and the regional fusion as claimed in claim 1, wherein a method for calculating the concave points of the chromosome regions in the step 6 is as follows:
1) as mentioned in the step 6, for any non-single chromosome regional image P, extracting a coordinate sequence of an outline of the non-single chromosome region first and recording as B, and recording an ith outline coordinate as B(i), wherein the i is the subscript index value;
2) initializing variables K=2, MAXSTEP=7 and i=1, wherein the MAXSTEP represents a maximum step length;
3) traversing the coordinate sequences of the outlines of the chromosome regions in sequence; and for a position of the ith outline coordinate, calculating a concave angle θ at the position of the ith outline coordinate, with a cosine value as follows:
$$cos ( θ ) = \frac{\overline{B ( i ) B ( i - K )} • \overline{B ( i ) B ( i + K )}}{\vert \overline{B ( i ) B ( i - K )} \vert \overline{\vert B ( i ) B ( i + K )} \vert}$$
where, the B̅(̅i̅)̅B̅(̅i̅−̅K̅)̅ represents a vector from an ith point B(i) to an (i−K)th point B(i−K) on the outline; and B̅(̅i̅)̅B̅(̅i̅+̅K̅)̅ represents a vector from the ith point B(i) to an (i+K)th point B(i+K) on the outline;
4) marking an outline point having the concave angle θ<T1 and a midpoint between B(i−K) and B(i+K) out of the chromosome region as a candidate concave point;
5) i=i+1; if the i is greater than the number of outline points, executing the step 6); or otherwise, continuing the steps 3) and 4);
6) K=K+1; if the K is greater than the MAXSTEP, executing the step 7); or otherwise, re-initializing i=1, and continuing the steps 3), 4) and 5);
7) marking the outline point between two candidate concave points with a distance smaller than 5 pixels along the outline as the candidate concave point; and
8) determining a midpoint of a candidate concave point segment: considering two candidate concave points having the distance of 1 pixel along the outline as being in a same concave point segment, and finding the midpoint of the concave point segment to serve as a final outline concave point.

5. The method for segmenting the G-banded adhered chromosome based on the geometrical characteristic and the regional fusion as claimed in claim 4, wherein in the step 1), the method for extracting the coordinate sequence of the outline is obtained with a relevant Application

6. The method for segmenting the G-banded adhered chromosome based on the geometrical characteristic and the, regional fusion as claimed in claim 1, wherein in the step 9, a method of the adhered chromosome segmentation strategy is as follows:
1) as mentioned in the steps 6-8, for any adhered chromosome regional image P, calculating the effective cutting line set CUT′, and the number n of the cutting lines;
2) respectively creating a set M and a set C having a length of n elements, wherein the type of each element in the set M is the image, the type of each element in the set C is the cutting line, and the elements in the two sets are initialized as 0; initializing the variable i=1, and initializing two image sets Si′ and Ad′ to be null, wherein the Si′ represents a segmented single chromosome image set, and the Ad′ represents a segmented non-single chromosome image set;
3) segmenting the image P into two portions P_{A} and P_{B} with the cutting line CUT(i);
4) determining whether the P_{A} and the P_{B} simultaneously meet the condition III and a condition V; if yes, the P_{A} and the P_{B} at this time being two segmented single chromosomes respectively, storing images of the two single chromosomes to the Si′ and executing the step 12); or otherwise, executing the step 5), wherein the condition V refers to that the skeleton line of the chromosome communicated regional image has two end points, the average value of the width for the chromosome region of the image is between [c1·Wₘᵢₙ,c2·Wₘₐₓ], and the c1 and the c2 are a correction factor;
5) determining whether the P_{A} meets the condition III or the condition V and the P_{B} also meets the condition III or the condition V; if yes, storing the cutting line at this time to the set C, and executing the step 7); or otherwise, executing the step 6);
6) determining whether either the P_{A} or the P_{B} meets the condition III and the condition V; and if yes, assigning an image meeting the conditions to the set M;
7) i=i+1 and determining whether the i is greater than n; if yes, executing the step 8); or otherwise, returning continuously to execute the steps 3), 4), 5) and 6);
8) if the set C is not null, a shortest cutting line in the set C being a final cutting line, segmenting the image P into two single chromosome portions with the shortest cutting line, storing the two single chromosome portions to the Si′, and executing the step 12); and if the C is null, executing the step 9);
9) determining whether the number of elements in the set M is equal to 1; if yes, the unique one element in the set M being the segmented single chromosome portion, storing an image of the single chromosome to the Si′, and executing the step 12); or otherwise, executing the step 10);
10) determining whether the number of elements in the set M is greater than 1; if yes, executing a fusion operation in the step 11); or otherwise, indicating that the adhered chromosome regional image P cannot be segmented effectively, and ending the adhesion segmentation procedure; and
11) carrying out the fusion operation on the elements in the set NI in pairs:
a. accessing kth and jth image elements M(k) and. M(j) (k≠j) in the M, wherein sizes of the two images are the same as the original adhered image P, and a chromosome region in each image is one portion of the P;
b. calculating an intersection area s1 between the chromosome regions in the M(k) and M(j) images, wherein the size of the intersection area is represented by the number of intersected pixels in the chromosome regions, and a sufficient and necessary condition for an existence of an intersection between the chromosome regions of the two images is that a pixel corresponding to a same coordinate position in the two images is within the chromosome regions;
c. calculating a minimum value s2 of the area of the chromosome region in each of the M(k) and M(j) images, wherein the area at each chromosome region refers to the number of pixels in the chromosome region,
d. if
$$\frac{s 1}{s 2} > T 2 ,$$
deleting an image having a small area of the chromosome region between the M(k) and the M(j) from the set M; or otherwise, accessing any other two image elements in the set M continuously, and repeating the above steps b, c and d; and
e after the steps b, c and d are executed completely, remaining elements in the set M being fused single chromosome images, storing these single chromosome images to the Si′, and executing the step 12);
12) storing the segmented single chromosomes in the Si′, removing these chromosome regions from the original chromosome regional image P and then storing remaining chromosome regional images to the Ad′; and
13) viewing the chromosome regions in the Ad as the non-single chromosomes, and repeating, the steps 6-9 continuously.

7. The method for segmenting the G-banded adhered chromosome based on the geometrical characteristic and the regional fusion as claimed in claim 4, wherein the T1=2.5, T2=0.78, T3=2, 14=0.8, T5=2.0, and correction factors c1=0.9, c2=1 1.

8. An apparatus for segmenting a G-banded adhered chromosome based on a geometrical characteristic and regional fusion, wherein the apparatus is configured to store or run a module, or the module is a constituent part of the apparatus, the module is a software module, one or more pieces of the software are provided, and the software module is configured to execute the method for segmenting the G-banded adhered chromosome based on the geometrical characteristic and the regional fusion as claimed in claim 1.

9. A chromosome karyotype analysis device, comprising an apparatus for segmenting an adhered image and an apparatus for carrying out karyotype analysis on a segmented chromosome, and the apparatus for segmenting the adhered image is the apparatus for segmenting the G-banded adhered chromosome based on the geometrical characteristic and the regional fusion as claimed in claim 8.

10. The apparatus as claimed in claim 8, wherein the software module is configured to execute the method for segmenting the G-banded adhered chromosome, based on the geometrical characteristic and the regional fusion as claimed in claim 2.

11. The apparatus as claimed in claim 8, wherein the software module is configured to execute the method for segmenting the G-banded adhered chromosome based on the geometrical characteristic and the regional fusion as claimed in claim 3.

12. The apparatus as claimed in claim 8, wherein the software module is configured to execute the method for segmenting the G-banded adhered chromosome based on the geometrical characteristic and the regional fusion as claimed in claim 4.

13. The apparatus as claimed in claim 8, wherein the software module is configured to execute the method for segmenting the G-banded adhered chromosome based on the geometrical characteristic and the regional fusion as claimed in claim 5.

14. The apparatus as claimed in claim 8, wherein the software module is configured to execute the method for segmenting the G-banded adhered chromosome based on the geometrical characteristic and the regional fusion as claimed in claim 6.

15. The apparatus as claimed in claim 8, wherein the software module is configured to execute the method for segmenting the G-banded adhered chromosome based on the geometrical characteristic and the regional fusion as claimed in claim 7.

16. The chromosome karyotype analysis device as claimed in claim 9, wherein the apparatus for segmenting the adhered image is the apparatus as claimed in claim 10.

17. The chromosome karyotype analysis device as claimed in claim 9, wherein the apparatus for segmenting the adhered image is the apparatus as claimed in claim 11.

18. The chromosome karyotype analysis device as claimed in claim 9. wherein the apparatus for segmenting the adhered image is the apparatus as claimed in claim 12.

19. The chromosome karyotype analysis device as claimed in claim 9, wherein the apparatus for segmenting the adhered image is the apparatus as claimed in claim 13.

20. The chromosome karyotype analysis device as claimed in claim 9, wherein the apparatus for segmenting the adhered image is the apparatus as claimed in claim 14.
